(19) United States
(12) Patent Application Publication
(10) Pub. No.: US 2017/0319146 A1
(43) Pub. Date: Nov. 9, 2017

(54) APPARATUS AND METHOD FOR EXTRACTING CARDIOVASCULAR CHARACTERISTIC

(71) Applicant: SAMSUNG ELECTRONICS CO., LTD., Suwon-si (KR)

(72) Inventors: Sang Yun PARK, Hwaseong-si (KR); Byung Hoon KO, Hwaseong-si (KR); Yong Joo KWON, Yongin-si (KR); Jae Min KANG, Seoul (KR); Youn Ho KIM, Hwaseong-si (KR); Seung Woo NOH, Seongnam-si (KR); Young Zoon YOON, Hwaseong-si (KR)

(73) Assignee: SAMSUNG ELECTRONICS CO., LTD., Suwon-si (KR)

(21) Appl. No.: 15/497,395

(22) Filed: Apr. 26, 2017

Related U.S. Application Data
(60) Provisional application No. 62/331,062, filed on May 3, 2016.

(30) Foreign Application Priority Data
Nov. 9, 2016 (KR) 10-2016-0149004

(51) Int. Cl. A61B 5/00; A61B 5/0225; A61B 5/022; A61B 5/021; A61B 5/02; A61B 7/04; A61B 5/16

(52) U.S. Cl. CPC A61B 5/7278; A61B 7/045; A61B 5/742; A61B 5/6843; A61B 5/681; A61B 5/165; A61B 5/0004; A61B 5/02233; A61B 5/02125; A61B 5/02116; A61B 5/02007; A61B 5/02255

(57) ABSTRACT

An apparatus for extracting a cardiovascular characteristic is provided. The apparatus may include: a first sensor configured to measure a vibration signal generated by a pulse wave of a subject; a second sensor configured to measure a pulse wave signal of the subject; a processor configured to perform an operation related to a cardiovascular characteristic on the basis of the measured vibration signal and pulse wave signal; and a main body in which the first sensor, the second sensor, and the processor are mounted.

### CROSS-REFERENCE TO RELATED APPLICATION(S)

This application claims priority under 35 USC §119(a) from Korean Patent Application No. 10-2016-0149004 filed on Nov. 9, 2016, in the Korean Intellectual Property Office, and claims the benefit of U.S. Provisional Patent Application No. 62/331,062 filed on May 3, 2016, in the U.S. Patent and Trademark Office, the entire disclosures of which are incorporated herein by reference for all purposes.

### BACKGROUND

### 1. Field

Apparatuses and methods consistent with exemplary embodiments relate to the extraction of a cardiovascular characteristic.

### 2. Description of Related Art

Pulse wave analysis (PWA) and pulse wave velocity (PWV) methods are typically used to non-invasively extract cardiovascular characteristics without the use of pressure cuffs. The PWA method is a method of extracting cardiovascular characteristics by analyzing the shape of a photoplethysmography (PPG) signal or of a body surface pressure signal from a peripheral part of the body, for example, a fingertip, a radial artery, or the like. The blood ejected from the left ventricle causes reflection at areas of large branches, such as the renal arteries and the iliac arteries, and the reflection affects the shape of the pulse wave or body pressure wave measured at the peripheral part of the body. Thus, by analyzing this shape, arterial stiffness, arterial age, aortic artery pressure waveform or the like can be inferred. The PWV method is a method of extracting cardiovascular characteristics by measuring a pulse wave transmission time. According to this method, a delay (a pulse transit time (PTT)) between an R-peak (left ventricular contraction interval) of an electrocardiogram (ECG) and a peak of a PPG signal or pressure pulse wave of a finger or the radial artery is measured by measuring the ECG and PPG signals of the peripheral part of the body and by calculating a velocity at which the blood from the heart reaches the peripheral part of the body by dividing an approximate length of the arm by the PPT.

Generally, in order to measure an ECG, a measurement position 1, which is the heart, and a measurement position 2 must form a closed loop through the body surface with the heart positioned at the center thereof, and thus, the right hand and the left hand should be simultaneously in contact with electrodes of a measurement system, or the electrodes of the system should be in contact with one point of the skin surface of the torso or chest at one side of the heart and also with another point at the other side of the heart. As a result, it is not easy to utilize measure an ECG using a personal wearable device.

### SUMMARY

This summary is provided to introduce a selection of concepts in a simplified form that are further described below in the Detailed Description. This summary is not intended to identify key features or essential features of the claimed subject matter, nor is it intended to be used as an aid in determining the scope of the claimed subject matter.

According to an aspect of an exemplary embodiment, there is provided an apparatus for extracting a cardiovascular characteristic, the apparatus including: a first sensor configured to measure a vibration signal generated by a pulse wave of a subject; a second sensor configured to measure a pulse wave signal of the subject; a processor configured to perform an operation to calculate a cardiovascular characteristic on the basis of the measured vibration signal and pulse wave signal; and a main body to which the first sensor, the second sensor, and the processor are mounted.

The apparatus may further include at least one strap connected to the main body and fixing the main body to the wrist by tension.

The first sensor may measure a vibration transferred to the main body through the at least one strap when the pulse wave is generated at a radial artery in a state in which the at least one strap is wrapped around the wrist by tension.

The first sensor may include a piezoelectric sensor configured to measure a vibration of the pulse wave transferred through the main body.

The first sensor may include a force sensor or a strain gauge configured to measure a contact pressure of the subject which is transferred though the main body.

The first sensor may include a piezo bender configured to generate an electrical signal according to deformation thereof, a rigid support body mounted in the main body so as to support both ends of the piezo bender in a state in which a cavity is formed between the rigid support body and the piezo bender, and a pressurizing block configured to receive a vibration of the pulse wave and to pressurize and thereby deform the piezo bender.

The first sensor may include a piezo bender configured to generate an electrical signal according to deformation thereof, a rigid support body mounted in the main body so as to support both ends of the piezo bender in a state in which a cavity is formed between the rigid support body and the piezo bender, and a force sensor configured to measure a contact pressure of the subject which is transferred through the main body and to receive the vibration of the pulse wave and to pressurize and thereby deform the piezo bender.

The main body may include a housing and the housing may accommodate the second sensor such that the second sensor is exposed to the subject.

The apparatus may include a stretchable connection part connecting the second sensor to the housing.

The housing may accommodate the first sensor therein such that the first sensor is be closer to the second sensor than to the processor.

The second sensor may include a sensor board, a light source mounted on the sensor board to emit light to the subject, and a detector mounted on the sensor board to detect light returning from the subject.

The light source may be one selected from a light emitting diode (LED), a laser diode, and a fluorescent body.

The processor may include a delay time calculator configured to calculate a delay time between the measured vibration signal and the pulse wave signal, and a cardiovascular characteristic extractor configured to extract a cardiovascular characteristic on the basis of the calculated delay time.

The processor may further include a feature point extractor configured to extract at least one of a peak point, a valley point, a maximum slope point, and a minimum slope point from each of the vibration signal and the pulse wave signal as feature points and a delay time calculator which calculates the delay time using the extracted feature points.

The apparatus may further include a communicator mounted in the main body and configured to transmit at least one of the vibration signal, the pulse wave signal, the feature points, the delay time, pulse wave velocity (PWV), and the cardiovascular characteristic to an external device.

The cardiovascular characteristic may include at least one of a blood pressure, vascular age, arterial stiffness, aortic pressure waveform, stress index and fatigue.

The apparatus may further include a display configured to provide the extracted cardiovascular characteristic to the user under a control of the processor.

The processor may include a preprocessor configured to preprocess the signals measured by the first sensor and the second sensor and a signal converter configured to perform an analog-to-digital conversion on the measured signals.

According to an aspect of another exemplary embodiment, there is provided an apparatus for extracting a cardiovascular characteristic, the apparatus including: a main body; at least one strap connected to the main body and configured to be wrapped around a wrist of a subject; a first sensor mounted in the main body and comprising a force sensor or a strain gauge configured to measure a contact pressure signal of the subject generated by a vibration that is transferred to the main body through the at least one strap when a pulse wave is generated; a second sensor mounted in the main body and configured to measure a pulse wave signal of the subject; and a processor mounted in the main body and configured to perform an operation to extract a cardiovascular characteristic on the basis of the contact pressure signal and the pulse wave signal.

The processor may separate the contact pressure signal into an alternating current (AC) component signal and a direct current (DC) component signal.

The processor may calculate a delay time between the AC component signal and the pulse wave signal and extract a cardiovascular characteristic on the basis of the calculated delay time.

The processor may collect information about a contact state of the force sensor on the basis of the DC component signal and determine to extract the cardiovascular characteristic on the basis of the collected information.

The processor may calibrate a value of the extracted cardiovascular characteristic or an estimation model that represents a relationship between the cardiovascular characteristic and the delay time on the basis of the DC component signal.

According to an aspect of another exemplary embodiment, there is provided an apparatus for extracting a cardiovascular characteristic, the apparatus including: a main body; at least one strap connected to the main body and configured to be wrapped around a wrist of a subject; a first sensor mounted in the main body and comprising a microphone configured to measure a sound wave signal generated by a vibration that is transferred to the main body through the at least one strap when a pulse wave is generated; a second sensor mounted in the main body and configured to measure a pulse wave signal of the subject; and a processor mounted in the main body and configured to perform an operation related to cardiovascular characteristic extraction on the basis of the sound wave signal and the pulse wave signal.

The microphone may include at least one of an electret microphone and a micro electro mechanical system (MEMS) microphone.

The first sensor may further include a diaphragm configured to convert a vibration signal transmitted to the main body through the at least one strap into the sound wave signal and transmit the sound wave signal to the microphone.

According to an aspect of another exemplary embodiment, there is provided a method of extracting a cardiovascular characteristic by a cardiovascular characteristic extracting apparatus comprising a main body in which a first sensor and a second sensor are mounted, the method including: measuring, at the first sensor, a vibration signal generated by a pulse wave of a subject; measuring, at the second sensor, a pulse wave signal; and performing an operation related to cardiovascular characteristic extraction on the basis of the vibration signal and the pulse wave signal.

The measuring of the vibration signal may include measuring the vibration signal transferred to the main body through at least one strap when a pulse wave is generated in a state where the at least one connected to the main body is wrapped around a wrist of a subject and fixes the main body to the wrist by tension.

The performing of the operation may include calculating a delay time between the vibration signal and the pulse wave signal and extracting a cardiovascular characteristic on the basis of the calculated delay time.

The performing of the operation may include extracting at least one of a peak point, a valley point, a maximum slope point, and a minimum slope point from each of the vibration signal and the pulse wave signal as feature points and the calculating of the delay time may include calculating the delay time using the extracted feature points.

The method may further include providing the extracted cardiovascular characteristic to a user through a display.

Other exemplary features and aspects will be apparent from the following detailed description, the drawings, and the claims.

### BRIEF DESCRIPTION OF THE DRAWINGS

The above and/or other exemplary aspects will become apparent and more readily appreciated from the following detailed description of exemplary embodiments, taken in conjunction with the accompanying drawings in which:

FIG. 1 is a schematic overall view of an apparatus for extracting a cardiovascular characteristic according to an exemplary embodiment.

FIG. 2 is a block diagram illustrating the apparatus for extracting a cardiovascular characteristic according to an exemplary embodiment.

FIG. 3 is a block diagram illustrating in detail an exemplary embodiment of the configuration of the processor of FIG. 2.

FIGS. 4A to 4C are graphs for describing signal processing procedures of the apparatus for extracting a cardiovascular characteristic.

FIG. 5 is a block diagram illustrating an apparatus for extracting a cardiovascular characteristic according to an exemplary embodiment.

FIG. 6A is a diagram illustrating a main body of an apparatus for extracting a cardiovascular characteristic according to an exemplary embodiment.

FIG. 6B is a diagram for describing a first sensor of the apparatus for extracting a cardiovascular characteristic.

FIG. 7 is a diagram illustrating a configuration of a main body of an apparatus for extracting a cardiovascular characteristic according to an exemplary embodiment.

FIGS. 8A and 8B are diagrams illustrating a configuration of a main body of an apparatus for extracting a cardiovascular characteristic according to an exemplary embodiment.

FIG. 9 is a diagram for describing an apparatus for extracting a cardiovascular characteristic according to an exemplary embodiment.

FIG. 10 is a flowchart illustrating a method of extracting a cardiovascular characteristic according to an exemplary embodiment.

FIG. 11 is a detailed flowchart showing procedures of performing a cardiovascular characteristic-related operation of the method of extracting a cardiovascular characteristic according to an exemplary embodiment.

Throughout the drawings and the detailed description, unless otherwise described, the same drawing reference numerals will be understood to refer to the same elements, features, and structures. The relative size and depiction of these elements may be exaggerated for clarity, illustration, and convenience.

### DETAILED DESCRIPTION

The following detailed description is provided to assist the reader in gaining a comprehensive understanding of the methods, apparatuses and/or systems described herein. Various changes, modifications, and equivalents of the systems, apparatuses and/or methods described herein will suggest themselves to those of ordinary skill in the art. In the following description, a detailed description of known functions and configurations incorporated herein will be omitted when it may obscure the subject matter with unnecessary detail.

It will be understood that, although the terms first, second, etc. may be used herein to describe various elements, these elements should not be limited by these terms. These terms are only used to distinguish one element from another. Also, the singular forms are intended to include the plural forms as well, unless the context clearly indicates otherwise. In the specification, unless explicitly described to the contrary, the word “comprise” and variations such as “comprises” or “comprising”, will be understood to imply the inclusion of stated elements but not the exclusion of any other elements. Terms such as “ . . . unit” and “module” denote units that process at least one function or operation, and they may be implemented by using hardware, software, or a combination of hardware and software.

Hereinafter, exemplary embodiments of an apparatus and method for extracting a cardiovascular characteristic will be described in detail with reference to the accompanying drawings.

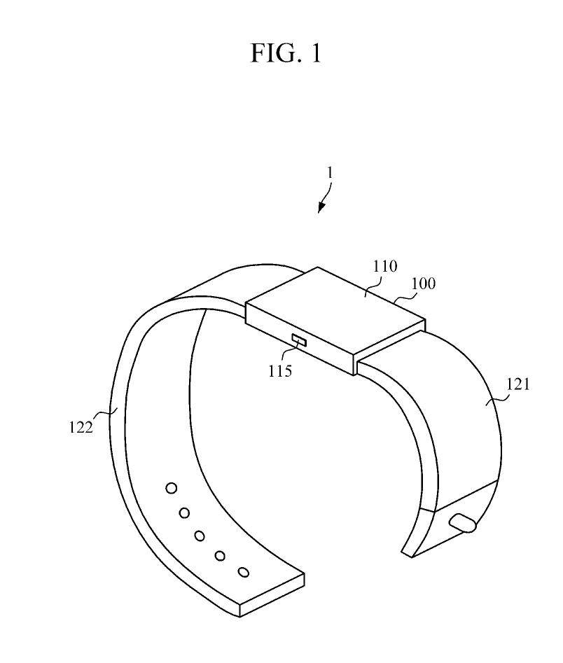

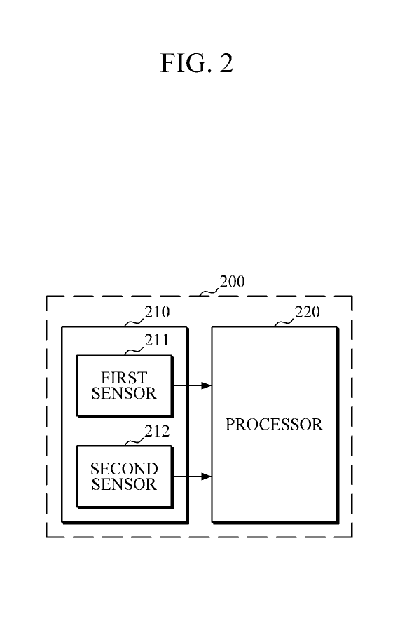

FIG. 1 is a schematic overall view of an apparatus for extracting a cardiovascular characteristic according to an exemplary embodiment. FIG. 2 is a block diagram illustrating the apparatus for extracting a cardiovascular characteristic according to an exemplary embodiment.

Referring to FIG. 1, an apparatus 1 for extracting cardiovascular characteristics according to an exemplary embodiment may be a wearable device which can be worn on a wrist. As shown in drawings, the apparatus 1 includes a main body 100 and straps 121 and 122 which are connected to the main body 100 and are flexible so as to be wrappable around and held against the wrist of a subject by tension, thereby fixing the main body to the wrist.

In addition, a display 110 and an operator 115 may be mounted on the main body 100 of the apparatus 1.

The display 110 may display a variety of information related to cardiovascular characteristics, for example, cardiovascular characteristic information, such as a blood pressure, vascular age, arterial stiffness, aortic pressure waveform, stress index and fatigue, various sensor signals used to extract the cardiovascular characteristics, analysis information of sensor signals, and additional information, such as warning and alarm according to an extracted cardiovascular characteristic, and may thereby provide this information to a user. In this case, the display 110 may provide the information to the user using any of a variety of predefined visual and/or non-visual methods. For example, if an extracted blood pressure corresponds to a dangerous level, the extracted blood pressure may be displayed in red, or if the extracted blood pressure corresponds to a normal level, the extracted blood pressure may be displayed in green.

Meanwhile, the display 110 may be equipped with a touch input function and may output a user interface so that the user can input any of a variety of commands using the touch input function and may thereby perform necessary operations.

The operator 115 may receive a control command of the user and transmit the command to a processor 220, and may include a power button for inputting a command for turning the power of the apparatus 1 on and off.

In addition, as shown in FIG. 2, the main body 200, according to an exemplary aspect may additionally include a sensor 210 and the processor 220.

The sensor 210 measures sensor signals required for extracting a cardiovascular characteristic. As shown in FIG. 2, the sensor 210 may include a first sensor 211 and a second sensor 212.

The first sensor 211 measures a first signal generated by a pulse wave of the subject. For example, when a pulse wave is generated at the radial artery in a state in which the straps 121 and 122 are wrapped around the wrist by tension, mechanical vibrations are transferred to the main body 100 through the straps 121 and 122. In this case, the first sensor 211 may measure a vibration signal transmitted to the main body 100 through the straps 121 and 122 as the first signal.

The first sensor 211 includes a sensor element for measuring the vibration of the radial artery pulse wave transferred to the main body 100 through the straps 121 and 122, and the sensor element may include a piezoelectric sensor having a piezoelectric characteristic that converts the mechanical vibration into an electric signal. For example, the first sensor 211 may be implemented with a piezo bender which is a plate piezoelectric sensor in which an electrical potential is generated according to the mechanical deformation of the plate, or may be implemented by serial and/or parallel connection of a plurality of piezo benders. In this case, the piezoelectric sensor may be include a piezoelectric ceramic, such as lead zirconate titanate (PZT), or a piezoelectric polymer, such as polyvinylidene fluoride (PVDF). However, this disclosure is not limiting, and the first sensor 211 may include any of various types of sensors capable of measuring a mechanical vibration of a pulse wave.

The second sensor 212 measures a second signal generated by the pulse wave of the subject. For example, the second sensor 212 may measure a pulse wave signal as the second signal by emitting light to the subject and detecting light returning from the subject. The second sensor 212 may be formed on the main body 100 so as to be in close contact with an area of the dorsal side of the wrist and may measure a pulse wave signal generated from capillary blood or venous blood. For example, the second sensor 212 may include a light source for emitting light to the subject and a detector for measuring the pulse wave signal by detecting light returning from the subject, which are sensor elements for measuring a pulse wave signal. In this case, the light source may include a light emitting diode (LED), a laser diode, or a fluorescent body, but is not limited thereto.

Meanwhile, the first sensor 211 and the second sensor 212 each may include an array of a plurality of sensor elements, if desired, for matching with the blood vessels in the wrist area and improving signal quality.

The processor 220 may receive the measured first and second signals and perform any of various operations related to extraction of cardiovascular characteristics using the received first and second signals.

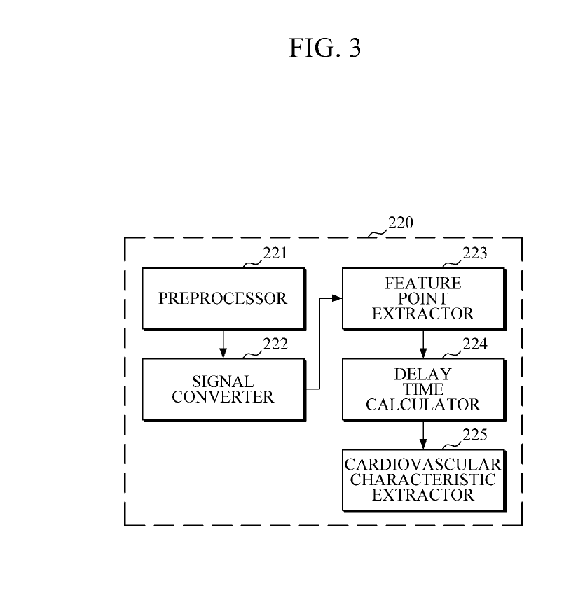

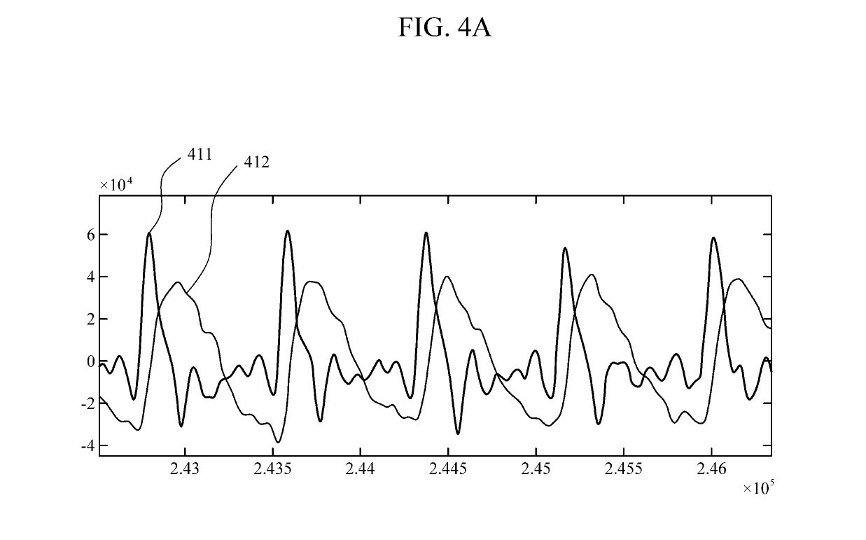

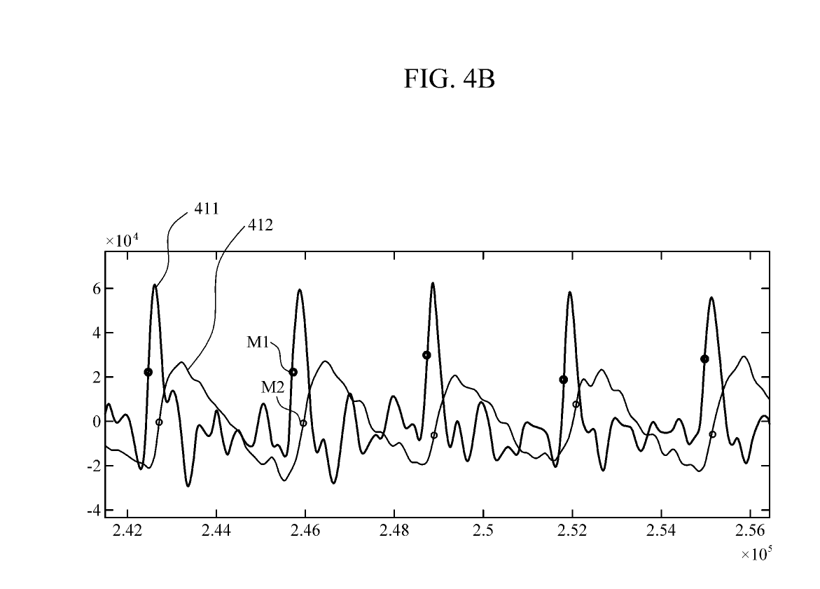

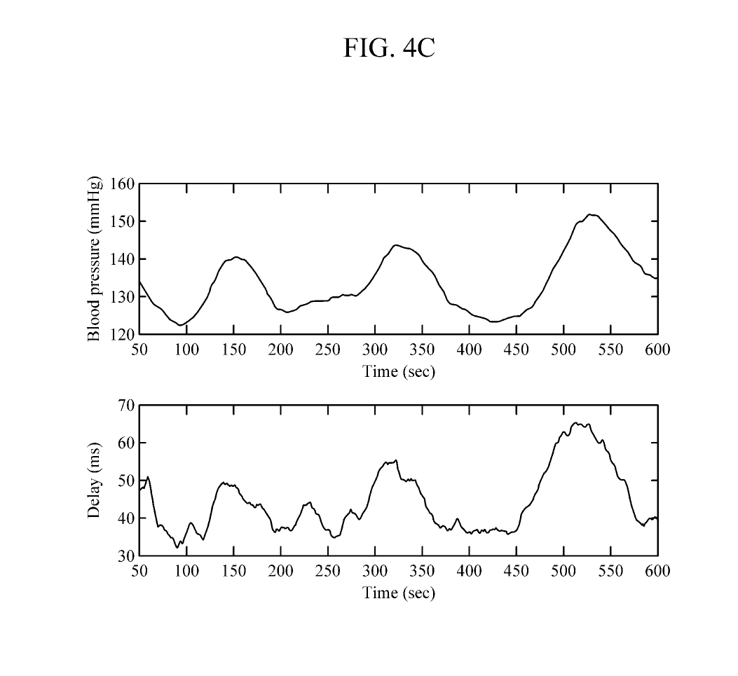

FIG. 3 is a block diagram illustrating in detail an exemplary embodiment of the configuration of the processor of FIG. 2. FIGS. 4A to 4C are graphs for describing signal processing procedures of the apparatus for extracting a cardiovascular characteristic.

As shown in FIG. 3, the processor 220 includes a preprocessor 221, a signal converter 222, a feature point extractor 223, a delay time calculator 224, and a cardiovascular characteristic extractor 225.

When the preprocessor 221 receives the first signal and the second signal from the first sensor 211 and the second sensor 212, respectively, the preprocessor 221 performs preprocessing on the received signals, such as noise removal, signal amplification, or the like. For example, the preprocessor 221 may perform preprocessing, such as signal normalization, detrending for trend and offset removal, signal smoothing, and high-frequency noise removal using a low pass filter.

In a case in which the measured first and second signals are analog signals, the signal converter 222 may convert the signals into digital signals.

FIG. 4A illustrates waveforms of signals obtained by analog-to-digital converting a first signal 411 measured by the first sensor 211 and a second signal 412 measured by the second sensor 212 at 1000 Hz and preprocessing the converted signals.

The feature point extractor 223 extracts feature points to obtain a delay time between the first signal 411 and the second signal 421 shown in FIG. 4A. For example, the feature point extractor 223 may extract notable points from the first signal 411 and from the second signal 412 as feature points and may place marks M1 and M2 on each of the signals 411 and 412, as shown in FIG. 4B, by using the extracted feature points or by combining the feature points. In this case, the feature points may include peak points, valley points, and/or maximum and minimum slope points of the first and second signals 411 and 412, but the exemplary embodiment is not limited thereto. FIG. 4B illustrates marking maximum points of the positive slopes of the first signal 411 and the second signal 412 as feature points.

The delay time calculator 224 may calculate a delay between the feature points M1 and M2 marked on the first signal 411 and on the second signal 412. The delay time indicates the time taken for a pulse of the radial artery to be applied to the dorsal side of the wrist. Since the first signal measured by the first sensor 211 is a mechanical vibration signal generated by the pulse wave of the radial artery and the second signal measured by the second sensor 212, for example, a pulse wave signal, is a pulse wave signal measured after arterial blood passing through the main blood vessels of the wrist and hand is applied thereto, the delay time between the mechanical vibration signal measured by the first sensor 211 and the pulse wave signal measured by the second sensor 212 may be assumed to have characteristics similar to those of a local pulse wave velocity (PWV) of peripheral parts of the body.

The cardiovascular characteristic extractor 225 may acquire a specific pattern on the basis of the delay time between the first signal and the second signal, where the delay time may be continuously calculated by the delay time calculator 224. In addition, the cardiovascular characteristic extractor 225 may extract a cardiovascular characteristic on the basis of the acquired delay time. In this case, the cardiovascular characteristic may include a blood pressure, vascular age, arterial stiffness, aortic pressure waveform, stress index and fatigue, but is not limited thereto.

The lower part of FIG. 4C illustrates a graph showing a continuously-acquired delay time between the first and second signals, and the upper part of FIG. 4C illustrates a graphs showing a systolic blood pressure when blood pressure changes are induced at several-minute intervals by breath holding or isometric exercise. Referring to FIG. 4C, it can be seen that there is a correlation between the systolic blood pressure and the pattern obtained from the delay time between the first signal and the second signal.

The cardiovascular characteristic extractor 225 may generate a correlation model that represents the correlation between the systolic blood pressure and the delay time between the first signal and the second signal. In this case, the correlation model may be generated, for example, in the form of a mathematic algorithm capable of inferring a blood pressure from the average delay time between the first signal and the second signal, but is not limited thereto, and may be stored in the form of a matching table in a storage device. In this case, the storage device may include a flash memory, a hard disk, a micro type multimedia card, a card type memory (e.g., SD or XD memory), a random access memory (RAM), a static random access memory (SRAM), a read only memory (ROM), an electrically erasable programmable read only memory (EEPROM), a programmable read only memory (PROM), a magnetic memory, a magnetic disk, or an optical disk, but is not limited thereto.

For example, the cardiovascular characteristic extractor 225 may measure and gather the first signal and the second signal as learning data for a predetermined period of time and then calculate the delay time between the first and second signals using the gathered learning data. In addition, the cardiovascular characteristic extractor 225 may measure a systolic blood pressure induced by breath holding or isometric exercise for a predetermined period of time and then generate a blood estimation formula as a correlation model which represents the correlation between the systolic blood pressure and the delay time calculated using the learning data.

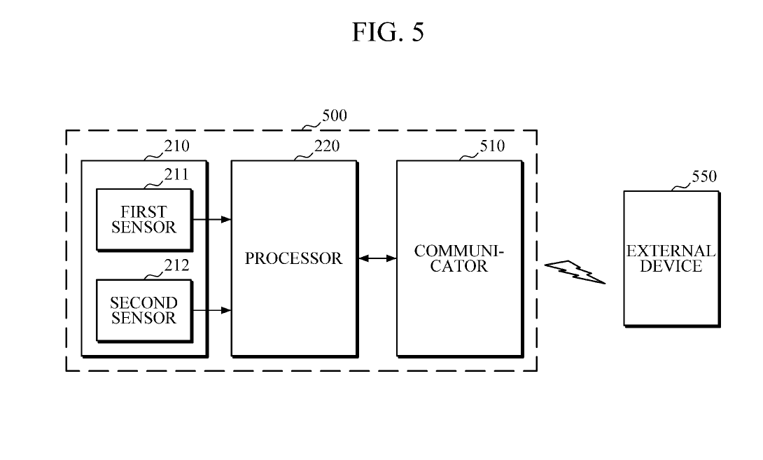

FIG. 5 is a block diagram illustrating an apparatus for extracting a cardiovascular characteristic according to an exemplary embodiment.

Referring to FIG. 5, in a main body 500 of the apparatus 1 includes a communicator 510, in addition to a sensor unit 210 and a processor 220.

As described above, the sensor unit 210 includes a first sensor 211 which measures a vibration signal generated by the pulse wave in the radial artery as a first signal and a second sensor 212 which measures a pulse wave signal of capillary blood or venous blood on the upper part of a wrist, wherein arterial blood passing from the radial artery through the main blood vessels in the wrist and hand is applied to the pulse wave signal.

The processor 220 processes any of various operations using the first signal and the second signal which are measured by the first sensor 211 and the second sensor 212, respectively. The processor 220 may control the communicator 510 to be connected to an external device 550 and may process any of various operations through collaboration with the connected external device 550. The processor 220 may provide information desired according to a degree of the cardiovascular characteristic extraction-related function, with which the associated external device 500 is equipped, to the external device 500. The information may include, for example, the measured first and second signals, a preprocessed signal, the calculated delay time, PWV, the correlation model, the extracted feature points, and/or the extracted cardiovascular characteristic information.

The communicator 510 may access a communication network by utilizing a communication technology under the control of the processor 220, and may be connected with the external device 550 accessing the same communication network and may thereby transmit and receive needed data. The communication technology may include Bluetooth communication, Bluetooth low energy (BLE) communication, near field communication (NFC), wireless local area network (WLAN) communication, ZigBee communication, infrared data association (IrDA) communication, Wi-Fi direct (WFD) communication, ultra-wideband (UWB) communication, Ant+ communication, Wi-Fi communication, radio frequency identification (RFID) communication, 3G communication, 4G communication, 5G communication, or the like, but is not limited thereto.

For example, when the cardiovascular characteristic is extracted based on the first signal and the second signal, the processor 220 may transmit the extracted cardiovascular characteristic to the external device 550 through the communicator 510 so that the cardiovascular characteristic is provided to the user through an interface module equipped in the external device 550, for example, a speaker, a display, a haptic device, or the like. The external device 550 may be a mobile terminal, such as a smartphone or a tablet personal computer (PC), which has superior computing performance relative to the apparatus 1 for extracting a cardiovascular characteristic, but is not limited thereto, and may include any of various types of information providing devices, such as a desktop PC, a notebook PC, or the like.

In another example, when the external device 550 has relatively excellent computing performance and is equipped with a function of extracting a cardiovascular characteristic using sensor signals, the communicator 510 may transmit the first signal measured by the first sensor 211 and the second signal measured by the second sensor 212 to the external device 550 under the control of the processor 220 to allow the external device 550 to perform the cardiovascular characteristic extraction. The external device 550 may be a device equipped with the cardiovascular characteristic extraction function, such as a smartphone, a tablet PC, a desktop PC, a notebook PC, a server, or the like. When the external device 550 receives a sensor signal from the communicator 510, the external device 550 may extract a cardiovascular characteristic and provide the cardiovascular characteristic to the user through the interface module equipped in the external device 550. In addition, when receiving the cardiovascular characteristic information from the external device 550 through the communicator 510, the processor 220 may provide the information to the user through a display.

In another example, when the processor 220 calculates the delay time between the first signal and the second signal and provides the delay time information to the external device 550, the external device 550 may use the correlation model and may extract a cardiovascular characteristic, such as a blood pressure of the user, using a correlation model managed by the external device 550 using the received delay time.

However, the collaboration between the apparatus 1 and the external device 550 is not limited to the above examples.

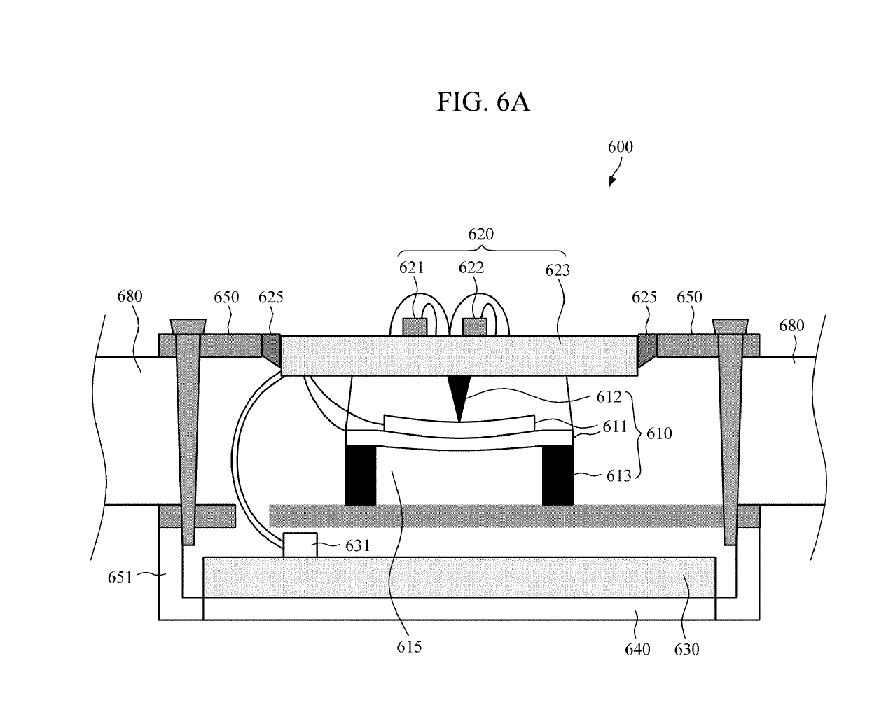

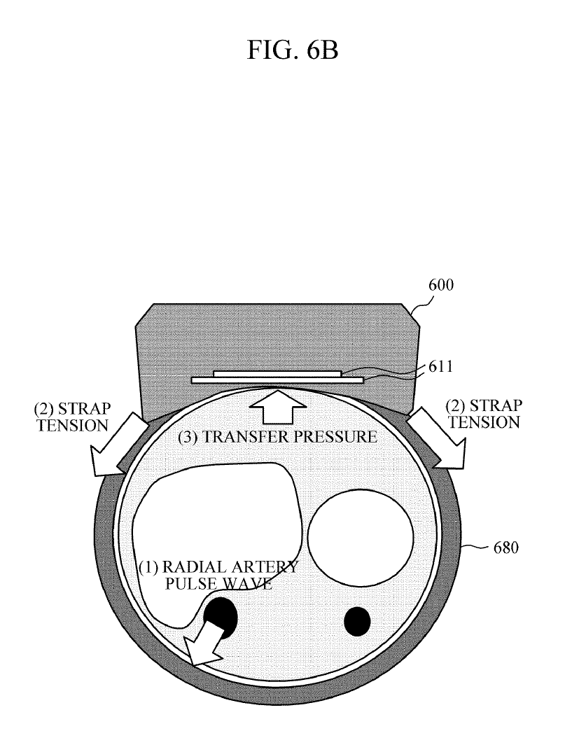

FIG. 6A is a diagram illustrating a main body of an apparatus for extracting a cardiovascular characteristic according to an exemplary embodiment. FIG. 6B is a diagram for describing a first sensor of the apparatus for extracting a cardiovascular characteristic.

Referring to FIG. 6A, the main body 600 of the apparatus 1 for extracting a cardiovascular characteristic according to the exemplary embodiment includes a first sensor 610, a second sensor 620, a main board 630, a display 640, and a housing 650.

The first sensor 610 is mounted in the housing 650. The housing 650 accommodates the first sensor 610 such that the first sensor 610 is relatively closer to the second sensor 620 than to the main board 630 on which the processor is mounted.

The first sensor 610 may include a piezo bender 611 which mechanically deforms according to a pressure applied thereto and which generates an electrical signal according to the deformation. The piezo bender 611 may be an array of two or more stacked layers as shown in the drawings. In this case, the piezo bender 611 may be electrically connected with a sensor board 623 of the second sensor 620, and the electrical signal generated by the piezo bender 611 may be transmitted to the sensor board 623 as a first signal. However, exemplary embodiments are not limited to the above description, such that the piezo bender 611 may be electrically connected with the main board 640 and the generated electrical signal may be directly transmitted to the main board.

The first sensor 610 may further include a pressurizing block 612 which generates the mechanical deformation of the piezo bender 611. In this case, the pressurizing block 612 may have one side in close contact with the sensor board 623 and the other side in close contact with the piezo bender 611. The pressurizing block 610 may pressurize the piezo bender 611 and cause the piezo bender 611 to deform according to the movement of the sensor board 623 when a mechanical vibration generated by the radial artery pulse wave (1) is transferred to the main body 600 through the tension of the straps (2) and in turn the movement occurs in the sensor board 623 (3). The pressurizing block 612 may be formed of a rigid material.

In addition, the first sensor 610 may further include a rigid support body 613 formed of a rigid material to support both ends of the piezo bender 611 in a state in which a cavity 615 is formed between the rigid support body 613 and the piezo bender 611.

The second sensor 620 is mounted in the housing 650 so as to be exposed to a subject, i.e., the dorsal side of a user's hand. The second sensor 620 includes a light source 621 and a detector 622, wherein the light source 621 is mounted on the sensor board 623 so as to be in contact with the subject and to emit light thereto, and the detector 622 is mounted on the sensor board 623 so as to detect light returning from the subject.

In addition, the second sensor 620 may further include the sensor board 623 having both ends connected with the housing 650. A connection part 625, disposed between the sensor board 623 and the housing 650, may be stretchable. When the mechanical vibration generated by the radial artery is transferred to the housing 650 of the main body 600 through straps 680, the stretchable connection part 625 of the housing 650 receives the mechanical vibration and generates a movement of the sensor board 623, and as a result, the pressurizing block 612 of the first sensor 610 deforms the piezo bender 611, as described above.

The sensor board 623 is electrically connected with the light source 621 and the detector 622, thereby transmitting a control signal of the processor to the light source 621 and receiving a measured second signal from the detector 622.

The main board 630 may be supported by a cover 651 and may include a display 640 on one surface thereof to provide a variety of information to the user. In addition, the above-described processor 220 may be mounted on the main board 630 and the processor 220 may perform various operations necessary for cardiovascular characteristic extraction on the basis of the first and second signals received through an element 631 electrically connected with the sensor board 623.

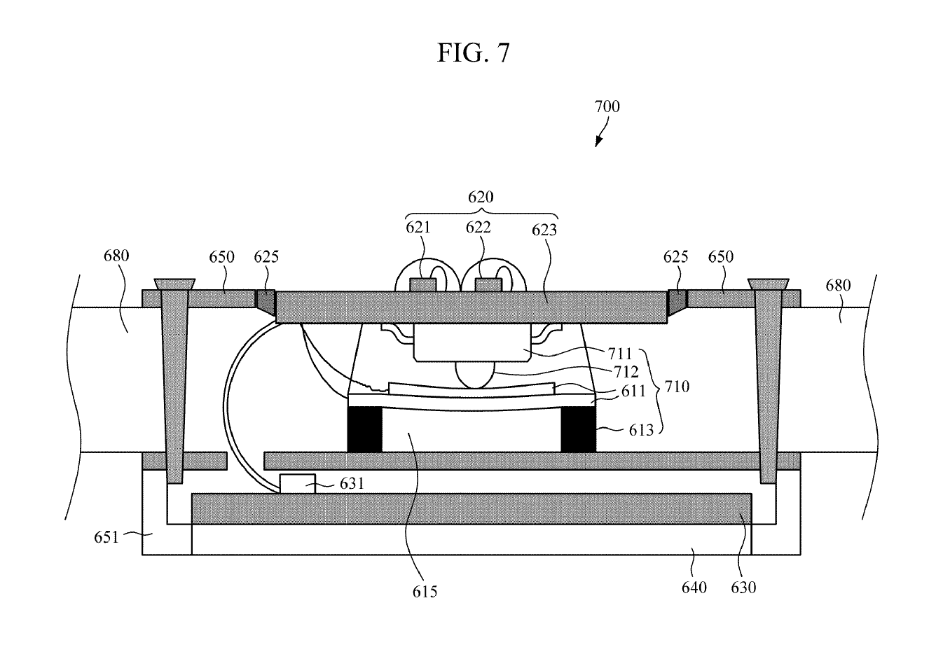

FIG. 7 is a diagram illustrating a configuration of a main body of an apparatus for extracting a cardiovascular characteristic according to an exemplary embodiment. The configuration according to the embodiment of FIG. 7 is an exemplary embodiment which modifies the first sensor of FIG. 6A.

Referring to FIG. 7, the configuration of the main body includes a first sensor 710, a second sensor 620, a main board 630, a display 640, and a housing 650. Features distinct from those discussed above with respect to FIG. 6A will be described.

The first sensor 710 is mounted in the housing 650 in a position such that the first sensor 710 is closer to the second sensor 620 than it is to the main board 630 on which the processor is mounted. The first sensor 710 includes a piezo bender 611 which mechanically deforms according to pressure and generates an electrical signal according to the deformation. In this case, the piezo bender 611 may be an array of two or more stacked layers as shown in the drawings. The piezo bender 611 may be electrically connected with the sensor board 623 of the second sensor 620, and the electrical signal generated in the piezo bender 611 is transmitted to the sensor board 623. However, this is not limiting, and alternately, the piezo bender 611 may be electrically connected with the main board 640, and the generated electrical signal may be directly transmitted to the main board 640.

In addition, unlike the embodiment of FIG. 6A, the first sensor 710 may include a force sensor. However, this is not limiting, and the first sensor 710 may include any of various other types of sensors for measuring a contact pressure, such as a strain gauge. The force sensor 711 is formed in close contact with the bottom part of the sensor board 623 and measures a contact pressure signal of the subject transmitted through the main body 700. In addition, the force sensor 711 receives a vibration of the pulse wave occurring in the radial artery through the straps and pressurizes and deforms the piezo bender 611. Thus, according to this exemplary embodiment, a pressurizing member 710 may be formed on the lower part of the force sensor 711 in order to pressurize the piezo bender 611.

The contact pressure signal measured by the force sensor 711 is transmitted to the sensor board 623 as a first signal along with a vibration signal measured by the piezo bender 611, and is transmitted to the main board 630 by the element which connects the sensor board 623 and the main board 630.

In addition, the first sensor 710 may further include a rigid support body 613 formed of a rigid material to support both ends of the piezo bender 611 in a state in which a cavity 615 is formed between the rigid support body 613 and the piezo bender 611.

The processor 220 formed on the main board 630 may perform any of various operations required to extract a cardiovascular characteristic on the basis of the first signal and the second signal received through the element 631 electrically connected with the sensor board 623. For example, the processor 220 may calculate a delay time on the basis of the vibration signal as the first signal and the pulse wave signal as the second signal and may extract the cardiovascular characteristic using the calculated delay time. Further, the processor 220 may monitor blood perfusion, compliance of capillaries, and the like using a direct current (DC) component of the first signal, which reflects the pressure exerted on the skin by the sensor, as the contact pressure signal.

The second sensor 620 is mounted in the housing 650 so as to be exposed to the dorsal side of the user's hand and includes a light source 621 and a detector 622 which are mounted on the sensor board 623. In addition, the second sensor 620 may further include the sensor board 623 having both ends connected with the housing 650 and may be formed to be stretchable so as to efficiently transfer the mechanical vibration generated by the pulse wave of the radial artery to the force sensor 711.

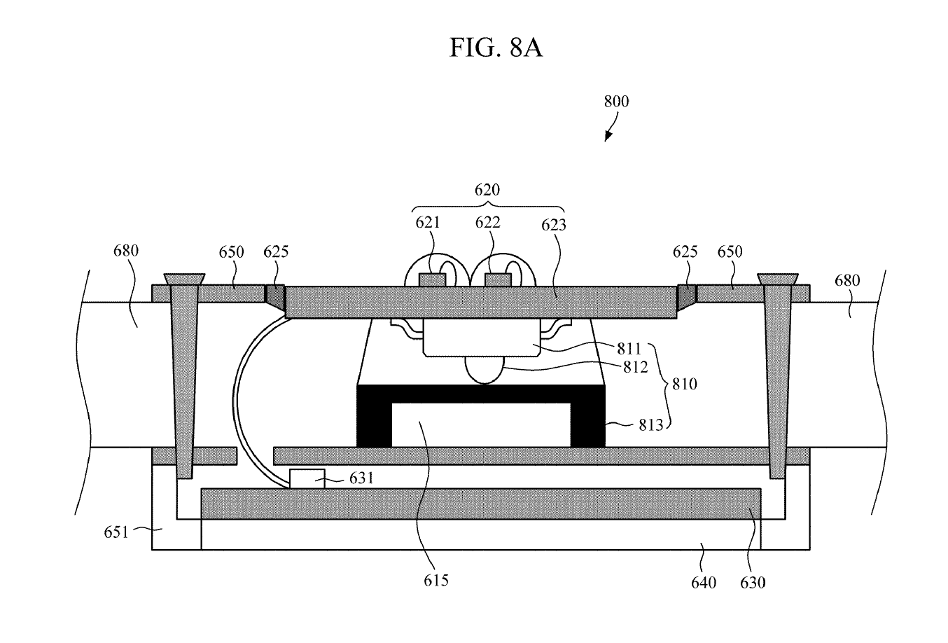

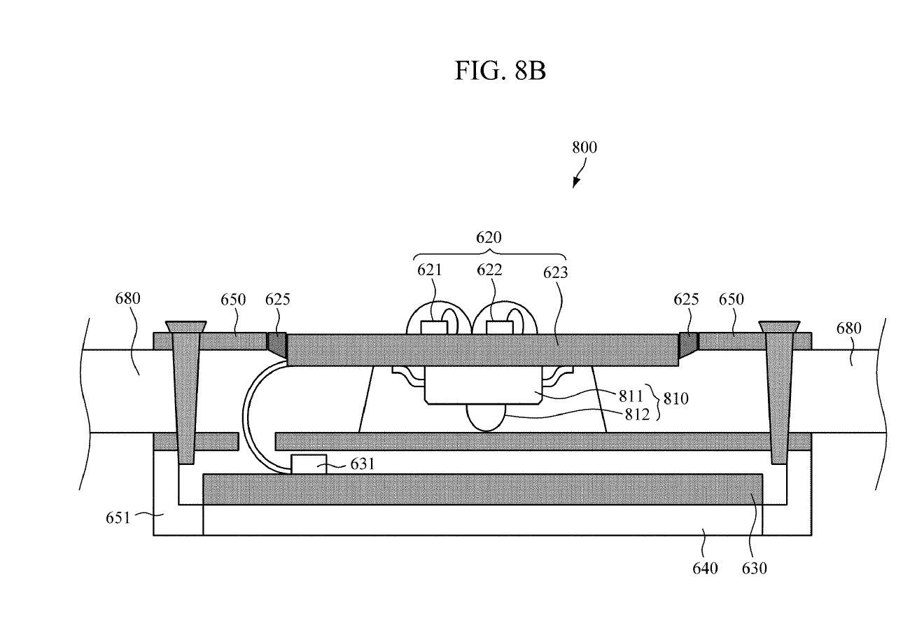

FIGS. 8A and 8B are diagrams illustrating a configuration of a main body of an apparatus for extracting a cardiovascular characteristic according to an exemplary embodiment. The configurations of the main body according to the embodiments of FIGS. 8A and 8B are exemplary embodiments which modify the first sensor of FIG. 7.

Referring to FIGS. 8A and 8B, the first sensor 810 may be realized by omitting the piezo bender 611 from the first sensor 710 of FIG. 7A. For example, as shown in FIG. 8A, the first sensor 810 may include a force sensor 811 with an appropriate sensitivity and a rigid support body 813 which supports the pressurization of a pressurizing member of the force sensor 811 in a state in which a cavity 615 is formed between the force sensor 811 and the rigid support body 813. The force sensor 811 is merely exemplary, and the sensor for measuring a contact pressure is not limited thereto. The force sensor 811 may include and of various other types of sensors capable of measuring a contact pressure, such as a strain gauge.

As described above, when the mechanical vibration generated by the pulse wave of the radial artery is transferred to the main body 800, particularly, to a sensor board 623 of a second sensor through straps, the force sensor 811 formed on the bottom part of the sensor board 623 may transmit a contact pressure signal as a first signal to the sensor board 623, wherein the contact pressure signal is generated as the pressurizing member 812 pressurizes the rigid support body 813.

When a processor 220 mounted in a main board 630 receives the contact pressure signal as the first signal, the processor 220 may separate the received first signal into an AC component signal and a DC component signal. In addition, the processor 220 may calculate a delay time between the two signals using the separated AC component signal and a pulse wave signal, which is a second signal measured by the second sensor 620, and may extract a cardiovascular characteristic on the basis of the calculated delay time.

In addition, the processor 220 may collect information on a contact state of the force sensor, which includes a contact pressure exerted on the subject by the force sensor 811, by using the separated DC component signal. The processor may then determine whether to extract the cardiovascular characteristic or re-measure the extracted signals using the measured signals on the basis of the collected information, and may determine whether to calibrate the extracted cardiovascular characteristic or an estimation model necessary for the cardiovascular characteristic.

For example, the main body 800 may be not be positioned at the exact examination point due to the thickness of the wrist on which the main body 800 is worn or the change of the wearing position due to the wearing position, the movement, or the like. In this case, the processor 220 may compare the contact pressure exerted on the subject by the force sensor 811 with a preset threshold. The processor 220 may perform cardiovascular characteristic extraction using the signals measured by the first sensor 810 and the second sensor 620 if the contact pressure is greater than or equal to the threshold, and otherwise, may determine that the measured signals are not accurate and control the first sensor 810 and the second sensor 620 to re-measure signals.

In addition, because the contact pressure generated by the force sensor 811 may affect one or more of blood perfusion and compliance of the capillaries, the processor 220 calculates the contact pressure on the basis of the DC component and calibrates the estimated cardiovascular characteristic value or the estimation model for representing the relationship between the delay time and the cardiovascular characteristic by applying the amount of change of blood perfusion or compliance of capillaries according to the calculated contact pressure to the estimated cardiovascular characteristic value or the estimation model. The estimation model may be a mathematical formula which determines the cardiovascular characteristic based on the delay time or a mapping table in which the delay time and the cardiovascular characteristic value are mapped to each other, but is not limited thereto.

Moreover, referring to FIG. 8B, according to this exemplary embodiment, the rigid support body 813 may be omitted from the first sensor 810 of FIG. 8A. Thus, according to the exemplary embodiment of FIG. 8B, the lower support part of a housing 650 which supports the first sensor 810 may be formed of a rigid material, and a force sensor 811 of the first sensor 810 may pressurize the lower support part of the housing 650 and measure a contact pressure signal. It is thus possible to reduce the volume of the main body configuration.

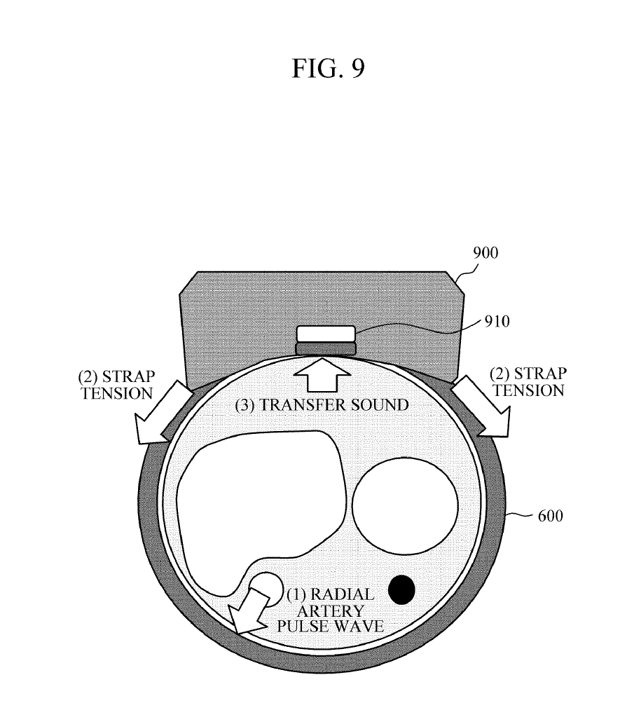

FIG. 9 is a diagram for describing an apparatus for extracting a cardiovascular characteristic according to an exemplary embodiment.

The apparatus for extracting a cardiovascular characteristic according to this exemplary embodiment includes a modification of the first sensor of FIG. 6A. It is assumed that the basic configuration of the main body 900, excluding the first sensor, is the same as the embodiment of FIG. 6A.

Referring to FIG. 9, the first sensor in the main body 900 includes a microphone 910. The microphone 910 may measure a sound wave signal (3) generated by the mechanical vibration which is transferred to the main body 900 through straps 680 (2) when a pulse wave is generated in the radial artery (1). The sound signal measured by the microphone 910 may be transmitted to a processor as a first signal. For example, the microphone 910 may include an electret microphone, a micro electro mechanical system (MEMS) microphone, or the like, but is not limited thereto.

In addition, the first sensor may further include a diaphragm in the front end thereof in order to convert the mechanical vibration signal transferred through the tension of the straps into the sound wave signal and transmit the sound wave signal to the microphone 910.

In this case, the processor 220 may calculate a delay time between the sound wave signal measured and transmitted by the microphone 910 and a pulse wave signal measured by the second sensor, and may extract a cardiovascular characteristic using the delay time.

Various modified embodiments of the configuration of the main body of the apparatus 1 for extracting a cardiovascular characteristic have been described with reference to FIGS. 6A to 9. However, these are merely exemplary and thus the aspects of the present disclosure are not limited to the above-described embodiments and various modifications may be made, as would be understood by one of skill in the art.

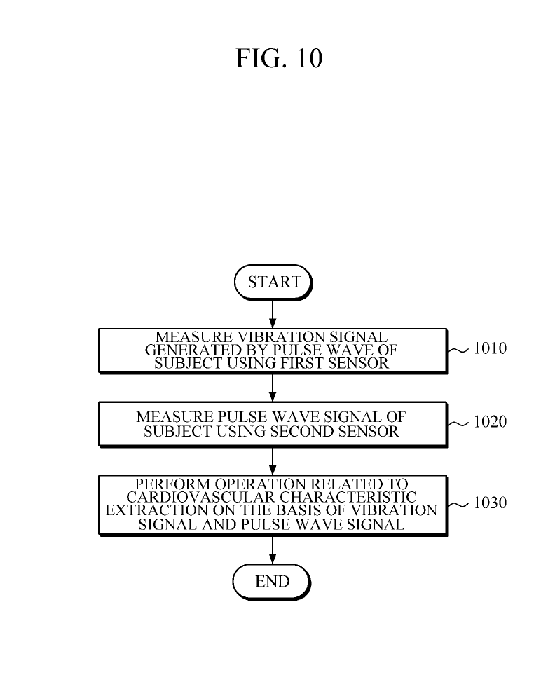

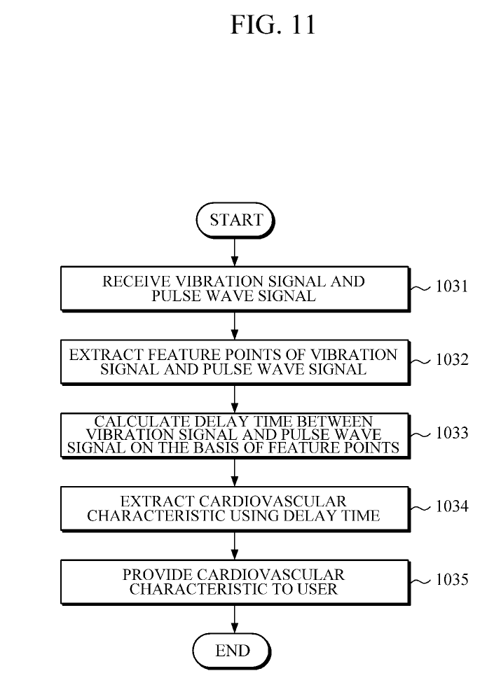

FIG. 10 is a flowchart illustrating a method of extracting a cardiovascular characteristic according to an exemplary embodiment. FIG. 11 is a detailed flowchart showing procedures of performing a cardiovascular characteristic-related operation of the method of extracting a cardiovascular characteristic according to an exemplary embodiment.

According to the exemplary embodiment of FIG. 10, the apparatus 1 for extracting a cardiovascular characteristic measures a first signal generated by a pulse wave of a subject by using a first sensor, as depicted in 1010. For example, when a pulse wave is generated at the radial artery in a state in which the straps of the apparatus 1 are wrapped around the wrist by tension and a mechanical vibration is transferred to a main body through the straps, the apparatus 1 may measure a vibration signal as the first signal. For example, the first sensor may include a piezoelectric sensor, such as a piezo bender, which deforms according to the mechanical vibration and generates an electrical signal.

The apparatus 1 measures a second signal generated by a pulse wave of the subject by using a second sensor, as depicted in 1020. For example, the second sensor may be formed on the main body so as to be in close contact with an area of the dorsal side of the wrist, and may measure a pulse wave signal generated from capillary blood or venous blood of the wrist by emitting light to the upper part of the wrist and detecting light returning therefrom.

Then, the apparatus 1 performs various operations related to cardiovascular characteristic extraction using the first and second signals, as depicted in 1030.

Operation 1030 will be described in detail with reference to FIG. 11. First, the first signal and the second signal are received from the first sensor and the second sensor, respectively, as depicted in 1031. In this case, preprocessing, such as detrending, signal smoothing, and high frequency noise removal using a low pass filter, and an analog-to-digital conversion may be performed on the first signal and the second signal, as desired.

Feature points are extracted from each of the received signals or each of the preprocessed signals, as depicted in 1032. The feature points may be notable points on the two signals used for calculating a delay time between the two signals, and may include, for example, peak points, valley points, and maximum and minimum slope points of the first and second signals, but are not limited thereto.

Then, when feature points are extracted from each of the signals, the delay time between the first signal and the second signal is calculated using the feature points, as depicted in 1033. The delay time is the time taken for a pulsation of the radial artery to be applied to the dorsal side of the wrist, and may be a factor that reflects a local PWV of peripheral parts of the body.

Thereafter, the user's cardiovascular characteristic is extracted using the calculated delay time, as depicted in 1034. It is possible to extract the cardiovascular characteristic which corresponds to the calculated delay time using a correlation model generated in advance. In this case, the correlation model may be generated in advance in the form of a mathematical formula or a matching table which represents a correlation between the delay time and a cardiovascular characteristic, such as a blood pressure.

The extracted cardiovascular characteristic is provided to the user through a display, as depicted in 1035. The extracted cardiovascular characteristic may be provided to the user in any of a variety of predefined visual and/or non-visual methods. For example, blood pressure information may be provided in a color corresponding to a range of an extracted blood pressure according to a user or a commonly applicable standard. In addition, appropriate guidance or warning information may be displayed according to a value of the extracted cardiovascular characteristic.

The current exemplary embodiments can be implemented as computer readable codes in a computer readable record medium. Codes and code segments constituting the computer program can be easily inferred by a skilled computer programmer in the art. The computer readable record medium includes all types of non-volatile record media in which computer readable data are stored. Examples of the computer readable record medium include a ROM, a RAM, a CD-ROM, a magnetic tape, a floppy disk, and an optical data storage. In addition, the computer readable record medium may be distributed to computer systems over a network, in which computer readable codes may be stored and executed in a distributed manner.

A number of examples have been described above. Nevertheless, it will be understood that various modifications may be made. For example, suitable results may be achieved if the described techniques are performed in a different order and/or if components in a described system, architecture, device, or circuit are combined in a different manner and/or replaced or supplemented by other components or their equivalents. Accordingly, other implementations are within the scope of the following claims.

What is claimed is:

1. An apparatus for extracting a cardiovascular characteristic, the apparatus comprising:
a main body, a first sensor mounted to the main body, a second sensor mounted to the main body, and a processor mounted to the main body; wherein:
the first sensor is configured to measure a vibration signal generated by a pulse wave of a subject;
the second sensor is configured to measure a pulse wave signal of the subject;
the processor is configured to calculate a cardiovascular characteristic based on the measured vibration signal and pulse wave signal.

2. The apparatus of claim 1, further comprising a first strap and a second strap connected to the main body, wherein the first strap and the second strap are configured to fix the main body to a wrist of the subject.

3. The apparatus of claim 2, wherein the first sensor measures a vibration transferred to the main body through the straps when the pulse wave is generated at a radial artery in a state in which the straps are wrapped around the wrist.

4. The apparatus of claim 1, wherein the first sensor comprises a piezoelectric sensor configured to measure a vibration of the pulse wave transferred through the main body.

5. The apparatus of claim 4, wherein the first sensor comprises one of a force sensor and a strain gauge configured to measure a contact pressure of the subject which is transferred though the main body.

6. The apparatus of claim 1, wherein the first sensor comprises a piezo bender configured to generate an electrical signal according to a deformation of the piezo bender, a rigid support body mounted in the main body which supports two ends of the piezo bender in a state in which a cavity is formed between the rigid support body and the piezo bender, and a pressurizing block configured to receive a vibration of the pulse wave and to pressurize and thereby deform the piezo bender.

7. The apparatus of claim 1, wherein the first sensor comprises a piezo bender configured to generate an electrical signal according to a deformation of the piezo bender, a rigid support body mounted in the main body which supports two ends of the piezo bender in a state in which a cavity is formed between the rigid support body and the piezo bender, and a force sensor configured to measure a contact pressure of the subject which is transferred through the main body and to receive the vibration of the pulse wave and to pressurize and thereby deform the piezo bender.

8. The apparatus of claim 1, wherein the main body comprises a housing and the second sensor is mounted within the housing such that the second sensor is exposed to the subject.

9. The apparatus of claim 8, further comprising a stretchable connection part connecting the second sensor to the housing.

10. The apparatus of claim 8, wherein the first sensor is disposed closer to the second sensor than to the processor.

11. The apparatus of claim 1, wherein the second sensor comprises a sensor board, a light source mounted on the sensor board and configured to emit light to the subject, and a detector mounted on the sensor board and configured to detect light returning from the subject.

12. The apparatus of claim 11, wherein the light source is one of a light emitting diode (LED), a laser diode, and a fluorescent body.

13. The apparatus of claim 1, wherein the processor comprises a delay time calculator configured calculate to a delay time between the measured vibration signal and the pulse wave signal, and a cardiovascular characteristic extractor configured to extract a cardiovascular characteristic based on the calculated delay time.

14. The apparatus of claim 13, wherein the processor further comprises a feature point extractor configured to extract at least one of a peak point, a valley point, a maximum slope point, and a minimum slope point from each of the vibration signal and the pulse wave signal as feature points and the delay time calculator is configured to calculate the delay time using the extracted feature points.

15. The apparatus of claim 14, further comprising a communicator mounted in the main body and configured to transmit at least one of the vibration signal, the pulse wave signal, the feature points, the delay time, pulse wave velocity (PWV), and the cardiovascular characteristic to an external device.

16. The apparatus of claim 13, wherein the cardiovascular characteristic comprises at least one of a blood pressure, a vascular age, an arterial stiffness, an aortic pressure waveform, a stress index, and fatigue.

17. The apparatus of claim 13, further comprising a display, wherein the processor is configured to control the display to provide the extracted cardiovascular characteristic to the user.

18. The apparatus of claim 13, wherein the processor comprises a preprocessor configured to preprocess the signals measured by the first sensor and the second sensor and a signal converter configured to perform analog-to-digital conversion of the measured signals.

19. An apparatus for extracting a cardiovascular characteristic, the apparatus comprising:
a main body;
at least one strap connected to the main body and configured to be wrapped around a wrist of a subject;
a first sensor mounted to the main body and comprising one of a force sensor and a strain gauge configured to measure a contact pressure signal of the subject generated by a vibration that is transferred to the main body through the at least one strap when a pulse wave is generated;
a second sensor mounted to the main body and configured to measure a pulse wave signal of the subject; and
a processor mounted to the main body and configured to calculate a cardiovascular characteristic based on the contact pressure signal and the pulse wave signal.

20. The apparatus of claim 19, wherein the processor separates the contact pressure signal into an alternating current (AC) component signal and a direct current (DC) component signal.

21. The apparatus of claim 20, wherein the processor calculates a delay time between the AC component signal and the pulse wave signal and extracts a cardiovascular characteristic based on the calculated delay time.

22. The apparatus of claim 21, wherein the processor collects information about a contact state of the force sensor based on the DC component signal and determines to extract the cardiovascular characteristic based on the collected information.

23. The apparatus of claim 21, wherein the processor calibrates one of a value of the extracted cardiovascular characteristic and an estimation model that represents a relationship between the cardiovascular characteristic and the delay time based on the DC component signal.

24. An apparatus for extracting a cardiovascular characteristic, the apparatus comprising:
a main body;
at least one strap connected to the main body and configured to be wrapped around a wrist of a subject;
a first sensor mounted to the main body and comprising a microphone configured to measure a sound wave signal generated by a vibration that is transferred to the main body through the at least one strap when a pulse wave is generated;
a second sensor mounted to the main body and configured to measure a pulse wave signal of the subject; and
a processor mounted to the main body and configured to calculate a cardiovascular characteristic based on the sound wave signal and the pulse wave signal.

25. The apparatus of claim 24, wherein the microphone comprises at least one of an electret microphone and a micro electro mechanical system (MEMS) microphone.

26. The apparatus of claim 24, wherein the first sensor further comprises a diaphragm configured to convert a vibration signal transmitted to the main body through the at least one strap into the sound wave signal and to transmit the sound wave signal to the microphone.

27. A method of extracting a cardiovascular characteristic by a cardiovascular characteristic extracting apparatus comprising a main body to which a first sensor and a second sensor are mounted, the method comprising:
the first sensor measuring a vibration signal generated by a pulse wave of a subject;
the second sensor measuring a pulse wave signal; and
calculating a cardiovascular characteristic based on the vibration signal and the pulse wave signal.

28. The method of claim 27, wherein the measuring of the vibration signal comprises measuring the vibration signal transferred to the main body through at least one strap when a pulse wave is generated in a state in which the at least one strap is connected to the main body and is wrapped around a wrist of a subject and fixes the main body to the wrist.

29. The method of claim 27, wherein the calculating the cardiovascular characteristic comprises calculating a delay time between the vibration signal and the pulse wave signal and extracting a cardiovascular characteristic based on the calculated delay time.

30. The method of claim 29, wherein the calculating the cardiovascular characteristic comprises extracting at least one of a peak point, a valley point, a maximum slope point, and a minimum slope point from each of the vibration signal and the pulse wave signal as feature points and the calculating the delay time comprises calculating the delay time using the extracted feature points.

31. The method of claim 29, further comprising providing the extracted cardiovascular characteristic to a user through a display.
